(19) United States
(12) Patent Application Publication
(10) Pub. No.: US 2002/0009498 A1
(43) Pub. Date: Jan. 24, 2002

(54) METHODS AND COMPOSITIONS FOR TREATMENT OF TRAUMATIC BRAIN INJURY

(76) Inventors: GUY L. CLIFTON, HOUSTON, TX (US); CARRIE G. MARKGRAF, HOUSTON, TX (US)

Correspondence Address: BAKER & BOTTS, 30 ROCKEFELLER PLAZA, NEW YORK, NY, 10112

(21) Appl. No.: 09/434,819

(22) Filed: Nov. 5, 1999

(51) Int. Cl. A61K 35/12

(52) U.S. Cl. 424/520

(57) ABSTRACT

The present invention provides methods and compositions for administration of nerve growth factor into subjects in whom the central nervous system has been damaged by trauma. The invention relates to formulations of nerve growth factor and their use in prevention of cognitive, motor or sensory deficits associated with traumatic injury of the brain or spinal cord. The invention further provides methods for treating such injuries comprising the administration of nerve growth factor formulations into the injured central nervous system. The present invention is based on the identification of novel optimal nerve growth factor dosages and treatment schedules to be used in the treatment of brain injured patients. In embodiments of the invention, nerve growth factor formulations are administered to subjects where the nervous system has been damaged by trauma to enhance the survival, growth or repair of neuronal cells.

### 1. INTRODUCTION

The present invention provides methods and compositions for administration of nerve growth factor into subjects in whom the central nervous system has been damaged by trauma. The invention relates to formulations of nerve growth factor and their use in prevention of cognitive, motor or sensory deficits associated with traumatic injury of the brain or spinal cord. The invention further provides methods for treating such injuries comprising the administration of nerve growth factor formulations into the injured central nervous system. The present invention is based on the identification of novel optimal nerve growth factor dosages and treatment schedules to be used in the treatment of brain injured patients. In embodiments of the invention, nerve growth factor formulations are administered to subjects where the nervous system has been damaged by trauma to enhance the survival, growth or repair of neuronal cells.

### 2. BACKGROUND OF INVENTION

Traumatic brain injury (TBI) affects nearly 200,000 people each year, most of them young men. Aggressive medical management has reduced the death rate, and currently, 75% of people survive a brain injury, but many are left with lasting cognitive and memory impairments that prevent their return to work or resumption of normal activities. Alterations in cognitive function remain a significant cause of long term morbidity after trauma to the central nervous system. Mild traumatic brain injury can result in cognitive deficits that are observed clinically and can also be seen following experimental brain injury models (Dacey et al., 1993, in Cooper PR (ed): Head Injury. Baltimore. Williams and Wilkins pp. 159-182; Hicks, 1993, J. Neurotrauma 10: 405-414).

One of the factors determining the degree to which elements of the central nervous system can recover from injury may be the availability of neurotrophic substances. Administration of various neuronal growth factors has been demonstrated to support neuronal cells in a variety of different models of central nervous system injury (Korsching S., 1993, J. Neurosci. 13:2739-2748; Maness et al., 1994, Neurosci. Biobehav. Rev. 18:143-159).

Nerve growth factor remains the most extensively studied neurotrophic factor, and treatment with nerve growth factor has been shown to reduce cell death after neuronal injury (Kerr, J F R et al., 1991, in Tomei DL, Cope/FO (eds): Apoptosis The Molecular Basis of Cell Death, Cold Spring Harbor, N.Y.: Cold Spring Harbor Press pp. 5-29; Frim D. M. et al., 1993, J. Neurosurg. 78: 267-273; Hagg T. et al., 1988, Exp. Neurol. 101: 303-312; Schumacher J. M. et al., 1991, Neuroscience 45: 561-570; Shigeno T. et al., 1991, J. Neurosci. 11: 2914-2919).

Additionally, nerve growth factor has been demonstrated to be a neurotrophic factor for forebrain cholinergic nerve cells that die during Alzheimer's disease and with increasing age (PCT Publication WO 90/07341). Nerve growth factor can also prevent the death of forebrain cholinergic nerve cells after traumatic injury and nerve growth factor has been reported to reverse the cognitive losses that occur with aging (Williams, Neurobiol. Aging 12:39-46(1991));Fisher, et al. 1991 J. Neurosci 11:1889-1906.

Goss et al., (1995, J Neurotrauma 12:159) have demonstrated increasing concentrations of nerve growth factor in the cortex after brain injury. Intraventricular infusion of NGF after cortical contusion or brain injury can significantly improve the cognitive deficits normally associated with fluid-percussion brain trauma (Sinson G. et al., 1995, J. Neurochem. 65:2209-2216; Dixon et al., 1991, J Neurosci Methods 39:253-262).

In addition, NGF has been reported to promote regeneration of sensory axons in a adult rat spinal cord and prevent death of central nervous system neurons after spinal cord lesions (Oudega M and Hagg T, 1996, Experimental Neurology 140:218-229; Diener P S and Bregman B S, 1994 NeuroReport 5:1913-1917).

While intravenous application of certain nerve growth factors for the treatment of neuronal damage associated with ischemia, hypoxia or neurodegeneration has been described, the usefulness of such therapies is questionable given the presence of the blood brain barrier which prevents exposure of the damaged neuronal tissue to the intravenously administered nerve growth factor (PCT Publication Number WO 90/0882). It has also been reported that nerve growth factor can be infused into the brain for treating neurodegenerative disorders, such as Parkinson's disease, Alzheimer's disease or Amyotrophic Lateral Sclerosis (ALS) by means of an implantable pump as described in PCT Publication Number WO 98/48723. In addition, nerve growth factor microencapsulation compositions having controlled release characteristics for use in promoting nerve cell growth, repair, survival, differentiation, maturation or function are described (PCT Publication Number WO 98/56426).

There is little known about the pharmacokinetics of nerve growth factor following intra-cerebral ventricular (i.c.v.) administration. It has been shown in rat and primate brain that nerve growth factor diffuses 2-3 mm from the site of i.c.v. administration. Despite these reports, nerve growth factor, administered by constant infusion into the ventricles of rats has been reported to significantly attenuate the cognitive deficits produced by TBI (Sinson G. et al., 1995, J. Neurochem. 65:2209-2216; Dixon et al., 1991, J Neurosci Methods 39:253-262). However, in rodents, at a “maximally effective dose” for stimulating basal forebrain activity, nerve growth factor produced weight loss due to anorexia, although the rats were otherwise healthy. The weight loss stopped when the nerve growth factor was discontinued (Williams, 1991, Experimental Neurology 113:31-37). In the limited human exposure to i.c.v. nerve growth factor, there were reports of weight loss due to anorexia and pain upon sudden movement, both of which resolved upon discontinuation of treatment (Seiger, A et al., 1993, Behavioral Brain Research 57:255-261; Ericsdotter, J M, 1998, Dement Geriatr Cogn Disord 9:246-257).

It is an objective of the present invention to provide novel optimized methods and compositions for treatment of human subjects in whom the nervous system has been damaged by trauma. The present invention provides optimal dosages of NGF that are higher than those previously disclosed for human use. In addition, the treatment schedules for administration of nerve growth factor into human subjects are of limited duration.

### 3. SUMMARY OF THE INVENTION

The present invention provides methods and compositions for administration of nerve growth factor into subjects in whom the nervous system has been damaged by trauma. The invention relates to formulations of nerve growth factor and their use in prevention of cognitive, sensory and motor deficits associated with traumatic central nervous system injuries. Such injuries include, for example, skull fractures, concussions, cerebral contusions and lacerations, intra-cranial hematomas, and neuronal damage resulting from infections or malignancies of the brain or spinal cord. The method of the invention comprises the infusion of nerve growth factor formulations directly into the central nervous system of the injured subject, for example, by infusion directly into the ventricles or spinal cord of the subject.

The results presented in the working examples herein demonstrate that administration of nerve growth factor following traumatic head injury can reduce the cognitive deficits associated with such injuries. The data obtained from the disclosed studies have been used in formulating an optimal range of dosages for use in humans. The present invention is based on the identification of optimal dosages of nerve growth factor and treatment schedules following traumatic head injury

### 4. BRIEF DESCRIPTION OF THE DRAWINGS

FIG. 1. Mean latency to reach to hidden platform for the 3 groups, Sham-operated and injured rats treated with nerve growth factor (0.3 μg/day i.c.v.) or CSF. The Sham group rapidly learned the platform's location as evidenced by the decreasing latency over trial blocks. The injured group treated with (cerebrospinal fluid) CSF did not. The injured group treated with nerve growth factor showed a significant improvement in their ability to learn the maze, but did not reach the level of the Sham group. * p<0.05 compared to the Sham group; * p<0.05 compared to the CSF group.

FIG. 2. Mean latency to stay on a balance beam on day 1 following TBI or Sham operation. Sham operated rats did not show a deficit in their ability to balance for 60 sec; all injured groups showed a deficit relative to the Sham controls but did not differ from one another. This pattern was maintained over the 7 days of neurologic recovery. * p<0.05 compared to the Sham group.

FIG. 3. The optimal dose of infusion of NGF is 0.3 μg/day. Nerve growth factor treatment was delivered over 14 days at 0.1 μg/day, 0.3 μg/day, or 0.9 μg/day. Both 0.1 μg/day and 0.3 μg/day improved cognitive abilities compared to the similarly treated CSF group. The 0.9 μg/day dose was toxic and the animals could not be tested. The full water maze test was given, but only the last trial block is shown. (*p<0.05 control group compared).

FIG. 4. The optimal duration of infusion for NGF (0.3 μg/day) is at least 28 days. Nerve growth factor treatment delivered over 7 days, 14 days or 28 days each improved cognitive abilities compared to the similarly treated CSF group. The full water maze test was given, but only the last trial block is shown. (*p<0.05 compared to CSF group).

FIG. 5. The optimal delay for administration of nerve growth factor at (0.3 μg/day for 14 days) can be up to 24 hours after TBI. Nerve growth factor treatment delayed 5 min, or 24 hours after TBI each significantly attenuated cognitive deficits compared to the similarly delayed CSF-treated group. Nerve growth factor administration delayed 7 days, however, had no effect on water maze performance. The full water maze test was given, but only the last trial block is shown here. * p<0.05 compared to CSF group.

### 5. DETAILED DESCRIPTION OF THE INVENTION

The present invention provides methods and compositions for administration of nerve growth factor into the central nervous system of subjects damaged by head and spinal cord trauma. The invention relates to formulations of nerve growth factor and their use in enhancement of neuronal cell survival, growth and repair in the central nervous system of injured subjects. The invention provides methods for attenuating cognitive deficits associated with traumatic brain injury wherein said methods comprise the administration of nerve growth factor directly into the central nervous system following traumatic head injury. The invention further provides methods for attenuating motor and sensory deficits associated with traumatic spinal cord injury wherein said methods comprise the administration of nerve growth factor directly into the central nervous system following traumatic spinal cord injury. Preferably, the nerve growth factor is infused into the central nervous system of the injured subject using, for example, a syringe or an infusion pump, although other methods that result in contact of the injured nervous system tissue with nerve growth factor may be used in the practice of the invention.

The methods of the invention provide a means for ameliorating cognitive, sensory or motor deficits through direct delivery of nerve growth factor to the central nervous system thereby avoiding the problems associated with the inability of systemically administered agents to cross the blood-brain-barrier. The present invention is based on the identification of optimal dosages of nerve growth factor and treatment schedules for administration of nerve growth factor following traumatic brain or spinal cord injury in human subjects. Subjects treated using the methods and compositions of the invention will have reduced cognitive, sensory and/or motor deficits due to nerve growth factor-mediated prevention of neuronal cell death normally associated with traumatic brain or spinal cord injury.

### 5.1. Compositions for Delivery of Nerve Growth Factor

The compositions for use in accordance with the present invention may be formulated in a conventional manner using one or more physiologically acceptable carriers or excipients in combination with nerve growth factor. Nerve growth factor refers to nerve growth factor from any species, including murine, bovine, ovine and preferably human, from any source including natural, synthetic or recombinantly produced. Preferably, the nerve growth factor is recombinantly produced wherein the nerve growth factor is cloned and its DNA expressed in, for example, mammalian, bacterial or yeast cells. In addition to naturally occurring nerve growth factor, modified forms of nerve growth factor having altered biological properties, such as increased affinity for nerve growth factor receptor, or increased bioavailability are also contemplated by the invention. Nerve growth factor is commercially available and may be obtained from manufacturers, such as Harlan Bioproducts, Inc. (Indianapolis, Ind.).

The nerve growth factor can be formulated in physiologically acceptable compositions suitable for delivery to the central nervous system. In general, the formulations may contain other components in amounts that do not detract from the preparation of effective safe formulations. For example, other physiologically acceptable excipients well known to those skilled in the art may be used. Optionally, formulations of nerve growth factor will contain physiologically acceptable carriers, preservatives buffers or stabilizers. Such formulations may include, for example, NGF dissolved in artificial CSF (ALZA Scientific Products). Acceptable carriers, excipients or stabilizers are nontoxic to recipients at the dosages and concentrations employed and will not detract from growth factor stability.

Particular formulations include liquid solutions or suspensions suitable for use with syringes, infusion pumps, such as implantable pumps, and catheters. Such formulations are well know to those skilled in the art. For liquid formulations the nerve growth factor can be dissolved in any recognized physiologically acceptable carrier for use in delivery of nerve growth factor formulations to the brain. The nerve growth factor formulation may further comprise nerve growth factor formulated in aqueous solution with a metal (in salt form) that binds nerve growth factor thereby stabilizing the nerve growth factor. Such metal salts include monovalent metals such as alkali metals, or alkaline earth metals or polyvalent metals such as zinc. The metal salt is not limited with the exception that it does not exert unwanted or deleterious influences in vivo.

### 5.2. Methods of Administration

The present invention relates to a method for attenuating cognitive, sensory or motor deficits associated with traumatic brain or spinal cord injury by administration of nerve growth factor into the injured central nervous system of a subject. The present invention is used as a prophylactic means to reduce the neuronal cell death associated with traumatic head or spinal cord injury. The method comprises the administration of nerve growth factor directly into the central nervous system of the injured subject soon after injury.

Compositions suitable for use in the present invention include compositions comprising nerve growth factor in a dose effective to achieve its intended purpose and one or more physiologically acceptable carriers. An effective dose refers to that amount of nerve growth factor sufficient to decrease neuronal cell death in the brain or spinal cord of the injured subject thereby attenuating the symptoms associated with traumatic brain or spinal cord injury. More specifically, an effective dose means an amount sufficient to reduce the neuronal cell death associated with brain or spinal cord injury that normally leads to cognitive, sensory and/or motor deficits in a traumatic brain or spinal cord injured patient. Determination of effective amounts is well within the capability of those skilled in the art. For example, the effective dose may be determined using a variety of different assays. The progress of the treated subject can be evaluated with clinical, neuropsychological, and neurophysiological tests well known to those skilled in the art. Such tests include those designed to measure memory functions such as the Hopkins Verbal Learning Test, the Wechsler Memory Scale-Revised and the ASIA Scoring System (Tator CH et al., 1995, in Clinical manifestations of acute spinal cord injury. Chapter 3 in contemporary management of spinal cord injury. Eds. Benzel E. C. et al., pp 15-26, American Assoc Neurol Surg, Park Ridge, Ill.).

The amount of composition administered is also dependent on the subject to whom the infused nerve growth factor is administered and the judgement of the physician overseeing the subject. It should be noted that the attending physician would know how and when to terminate, interrupt or adjust the treatment to a lower dose due to toxicity. Conversely, the attending physician would also know to adjust treatment to higher levels if the clinical response is not adequate. For example, the doses of nerve growth factor recommended, infra, are dependent on the volume of the cerebrospinal fluid in the brain and spinal cord, however, following injury the volume of the fluid may change as a result of the injury. In such instances, the attending physician would know how to adjust the dose appropriately. Changes in the doses of nerve growth factor can be determined by measurement of the memory, sensory or motor functions of the treated subject and/or by measurement of levels of nerve growth factor in the CSF.

In general, the dose range of nerve growth factor should be levels ranging between 150 μg and 4 mg delivered daily into the brain of the injured human subject. For example, a dose of between 250 μg and 3 mg of nerve growth factor may be infused daily, while most preferably the usual dose of nerve growth factor in the brain is between 500 μg and 1.5 mg daily dosing range.

The present invention relates to methods for prevention of cognitive deficits associated with neuronal cell death in brain injured subjects, therefore, the initial nerve growth factor treatment is administered shortly after the traumatic brain injury. In general, administration of nerve growth factor begins immediately after injury, i.e., within hours after injury, and up to Day-28 post-injury, while most preferably the treatment begins on Day 4-8 post-injury.

The treatment continues on a daily basis for between at least 10 and 112 consecutive days. For example, the treatment with nerve growth factor can continue on a daily basis for between at least 20 and 54 days, while most preferably the treatment continues for at least 28 consecutive days.

It is further recommended that infants, and children, receive lower doses, and they be titrated based on individual clinical response and volume of CFS space. Doses may be also be altered due to changes in the volume of cerebrospinal fluid in the treated subject. It may be necessary to use dosages outside the ranges disclosed above in some cases as will be apparent to those of ordinary skill in the art.

In general, the nerve growth factor is given as the sole neurotrophic agent if it is found to adequately control neuronal cell death. However, the nerve growth factor may be co-administered to a brain injured subject in combination with other neurotrophic reagents, including but not limited to, brain derived neurotrophic factor (BDNF) (Henderson, C. E. et al., 1993 Nature 363:277); neurotrophin-3(NT-3); ciliary neurotrophic factor (CNTF); and glial-derived neurotrophic factor (GDNF), to name a few.

Typically the nerve growth factor formulations will be administered intraventricularly or intrathecally using techniques known to those skilled in the art. The NGF may also be administered into the basal forebrain, hippocampus or the subarachnoid space. For treatment of spinal cord injuries the NGF may be administered into the subarachnoid space in close proximity to the site of spinal cord injury. For administration of nerve growth factor into the brain or spinal cord, the compositions for use according to the present invention are conveniently delivered in the form of liquid formulations administered via a syringe or a pump. In a specific embodiment of the invention, the nerve growth factor may be delivered to the brain of the subject by infusion of nerve growth factor compositions using an implanted pump and catheter, or shunt. In the use of a pump the dosage unit may be determined by providing a valve to deliver a metered amount. Commercially available pumps, including osmotic pumps or implantable pumps, may be used to deliver nerve growth factor into the brain of a subject. Such pumps include a Synchromed programmable pump (Medtronic P.L., Minneapolis, Minn.) or an Infusaid constant-rate infusion pump (Shiley Infusaid, Norwood, Mass.). Such pumps can be adjusted to provide a flow rate of, for example, 1-10 ml/day.

### 6. EXAMPLE

### Infusion of Nerve Growth Factor as Prophylaxis to Reduce Neurological Deficits Resulting From Traumatic Brain Injury

The following section describes experimental data relating to infusion of nerve growth factor into the brain following traumatic brain injury. As indicated by the data, the administration of nerve growth factor into the injured brain is capable of reducing cognitive deficits normally associated with traumatic brain injury. Treatment with nerve growth factor can be delayed up to 4 days and still retain efficacy. While infusion for 7 days is effective in treating the memory deficits, infusion for 14 days produces an effect of greater magnitude and infusion for 28 days increases the magnitude of the effect even more, so the longer the infusion the better, up to the point where toxicity is shown. In the current series of experiments, no adverse effects of nerve growth factor therapy could be detected.

### 6.1. Materials and Methods

Experimental traumatic brain injury (TBI) was produced using the controlled cortical impact model in anesthetized Sprague-Dawley rats (Dixon et al, 1997, Experimental Neurology 146:479-490). Following TBI, an osmotic mini-pump was implanted, subcranially, and attached to a brain infusion cannula placed stereotaxically in the left lateral ventricle. 7S Nerve growth factor (recombinant rat growth factor; Harlan Bioproducts) 0.3 μg/day at a rate of 0.5 μl/h) or artificial cerebral spinal fluid (CSF) was delivered for 14 days. On day 14, rats were given a cognitive memory test using the Morris water maze. The investigator who conducted the water maze test was blinded to the rats' group assignments.

To determine the effect of nerve growth factor on neurological deficits following TBI, rats underwent either sham surgery or TBI and were assigned to one of the following groups (n=12 each): SHAM, nerve growth factor (nerve growth factor at 0.3 μg/day i.c.v.); CSF (0.5 μl/day i.c.v.) or NONE (empty pump with a cannula i.c.v.). The NONE group was added to assess the effect of infusing any substance into the ventricle. Following TBI or Sham procedure and pump implantation, rats were tested on days 1, 2 and 7 using a standard battery of neurologic tests. The investigator who conducted these tests remained blinded to the rats' group assignments.

To determine the dose-response relationship for nerve growth factor administration and cognitive deficits, Sham and TBI-nerve growth factor treated groups were compared to TBI-CSF treated groups at each of the doses listed in Table I below. Water maze testing was carried out on day 14 after injury by an experimenter who was blinded to the group assignments of the rats.

TABLE I
GROUPS AND TREATMENTS FOR THE DOSE-RESPONSE STUDY

| STUDY | n | DOSE OR VOLUME | TOTAL NGF |
|---|---|---|---|
| LOW DOSE | | | |
| Sham | 12 | — | |
| NGF | 12 | 0.1 μg/day | 1.4 μg |
| CSF | 12 | 0.5 μl/h | |
| MEDIUM DOSE | | | |
| Sham | 12 | — | |
| NGF | 12 | 0.3 μg/day | 4.2 μg |
| CSF | 12 | 0.5 μl/h | |
| HIGH DOSE | | | |
| Sham | 12 | — | |
| NGF | 12 | 0.9 μg/day | 12.6 μg |
| CSF | 12 | 0.5 μl/h | |

To determine the optimal length of infusion, a sham operation or TBI was performed using the controlled cortical impact method, and, immediately following injury, an osmotic pump was implanted subcranially, with a cannula in the left lateral ventricle for delivery of nerve growth factor or CSF. Cognitive performance was assessed using the water maze task on the last day of nerve growth factor or CSF treatment.

### 6.2. Results

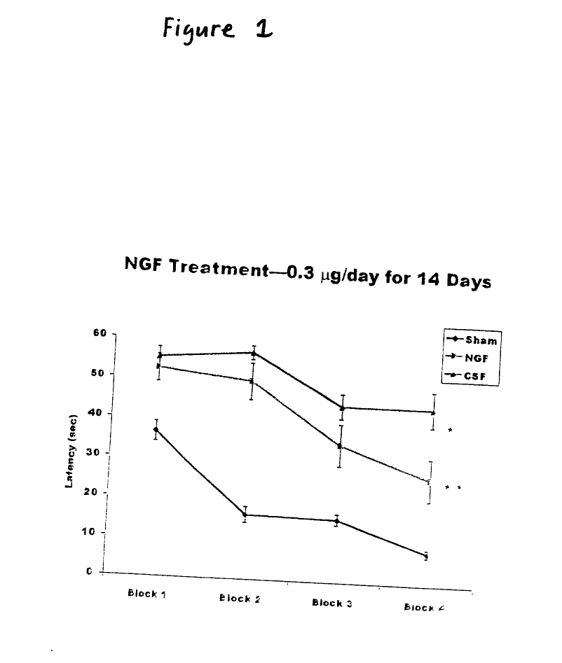

FIG. 1 shows that nerve growth factor infused into the ventricles of rats for 14 days following TBI significantly improves memory deficits compared to vehicle-treated controls. Rats (n=12/group) received either SHAM or TBI surgery. The SHAM group rapidly learned the platform location as evidenced by the decreasing latency over trial blocks. The injured group treated with CSF did not. The injured group treated with nerve growth factor showed a significant improvement in their ability to learn the maze.

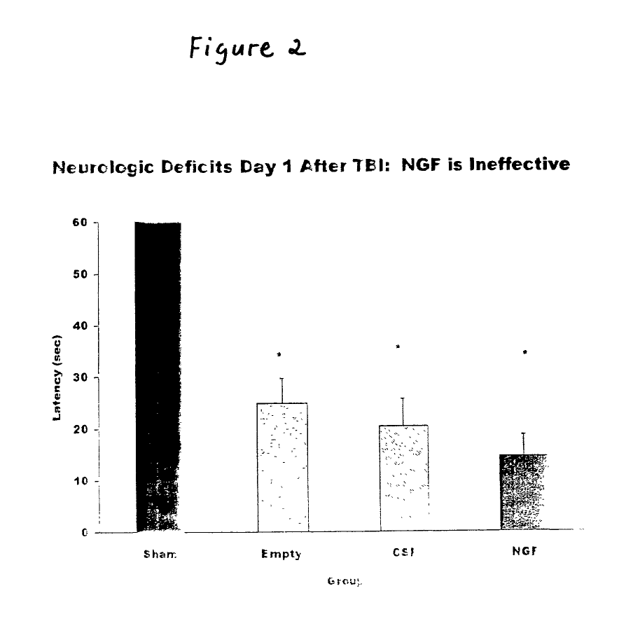

Nerve growth factor, did not have an effect on early neurologic deficits following cortical impact injury in rats as measured by the ability of the rats to stay on a balance beam. As shown in FIG. 2, nerve growth factor had no effect on neurologic behavior after injury. Sham operated animals did not show a deficit in their ability to balance for 60 sec; all injured groups showed a deficit relative to the Sham controls but did not differ from one another.

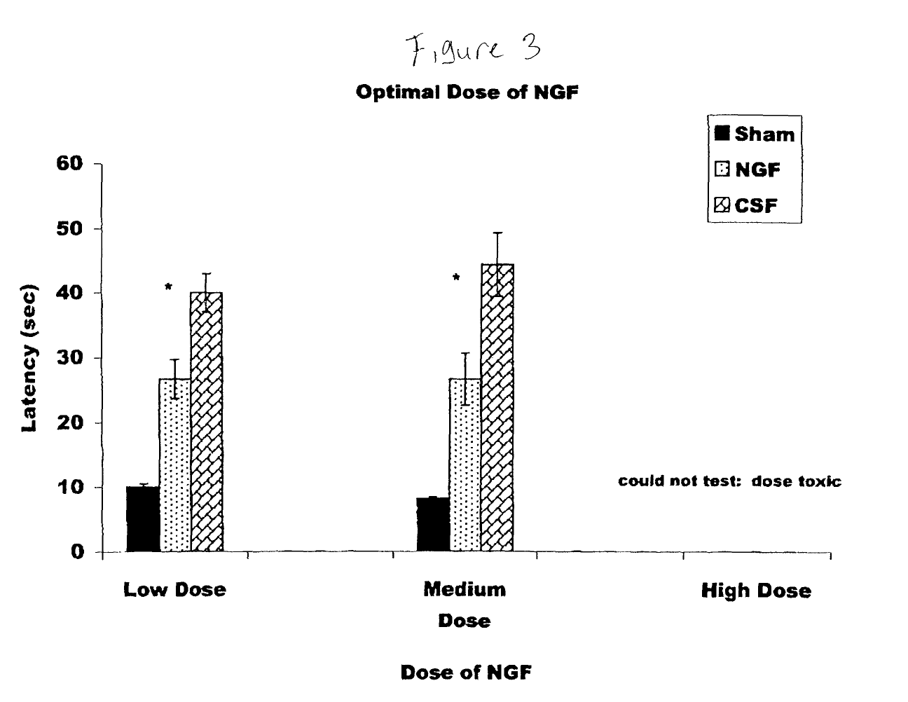

A dose-response study showed that i.c.v. administered nerve growth factor was effective at doses of 0.1 μg/day and 0.3 μg/day but not at 0.9 μg/day, when each of these doses was infused for 14 days after TBI. As FIG. 3 shows, both the 0.1 μg/day and 0.3 μg/day doses were effective in attenuating the cognitive deficits following TBI. 0.9 μg/day dose was ineffective, possibly due to the side effects seen at this dose. The lower dose was less effective than the higher dose.

Nerve growth factor at 0.1 μg/day for 14 days significantly attenuated the cognitive deficits as measured in water maze performance compared to the CSF-treated group. The nerve growth factor treated animals were significantly different from the Sham group as well, indicating that they did not learn as well as the Shams. Nerve growth factor at 0.3 μg/day for 14 days significantly attenuated the cognitive deficits seen in water maze performance compared to CSF-treated group. The magnitude of the effect was larger than that seen in the low dose study. The nerve growth factor-treated rats were significantly different from the Sham group as well, indicating that they did not learn well.

Optimal length of infusion for nerve growth factor was evaluated for 3 time periods: infusion for 7 days, 14 days and 28 days. Each treatment group was compared to a cerebral spinal fluid (CSF)-treated group of an identical length, and a Sham-operated control group, as indicated in Table II below.

TABLE II

| STUDY | n | DOSE OR VOLUME | TOTAL NGF |
|---|---|---|---|
| 7 DAY INFUSION | | | |
| Sham | 12 | — | |
| NGF | 12 | 0.3 μg/day | 2.1 μg |
| CSF | 12 | 0.5 μl/h | |
| 14 DAY INFUSION | | | |
| Sham | 12 | — | |
| NGF | 12 | 0.3 μg/day | 4.2 μg |
| CSF | 12 | 0.5 μl/h | |
| 28 DAY INFUSION | | | |
| Sham | 12 | — | |
| NGF | 12 | 0.3 μg/day | 8.4 μg |
| CSF | 12 | 0.25 μl/h | |

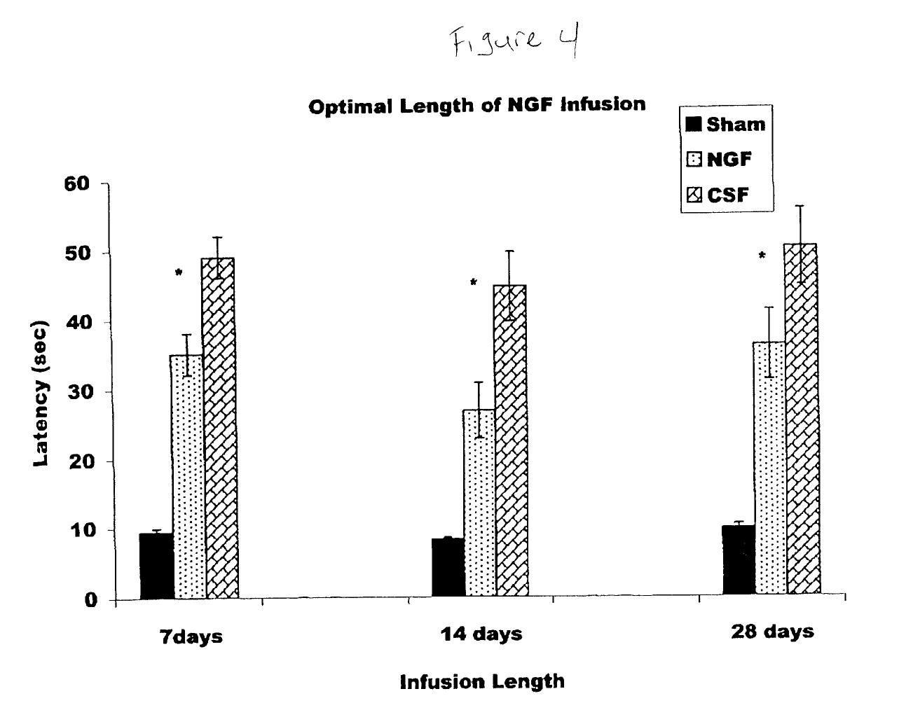

As FIG. 4 shows, each of the tested infusion lengths were effective in attenuating cognitive deficits, but the longer the infusion, the greater the magnitude of the improvement.

Nerve growth factor at 0.3 μg/day for 7 days significantly attenuated cognitive deficits compared to the CSF-treated group. The magnitude of the effect was greater at 14 days and 28 days than that seen for 7 days (FIG. 4).

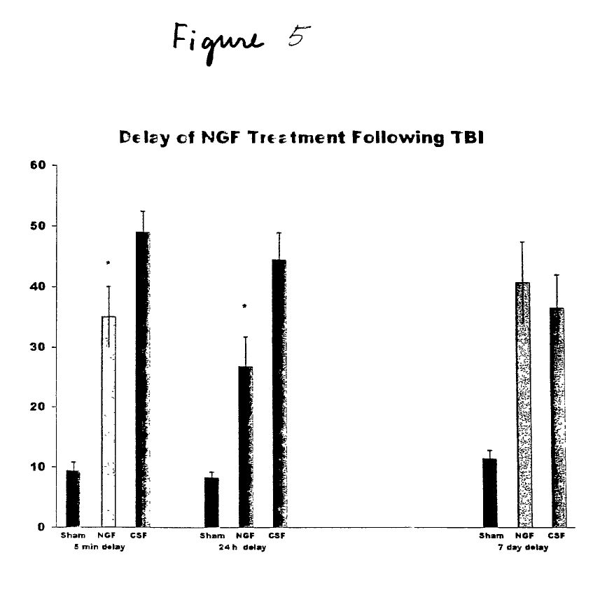

Treatment with nerve growth factor can be delayed up to 4 days after injury and still retain efficacy in improving cognitive performance. Studies examined the effect of i.c.v. nerve growth factor (0.3 μg/day for 14 days) with the following delays: 5 min., 24 h., 4 days and 7 days. Each of the nerve growth factor-treated groups was compared to an injured-CSF treated group delayed by the same interval, and to a Sham operated group (n-12 each). Cognitive abilities were assessed using water maze performance on the last day of nerve growth factor or CSF treatment. As indicated in FIG. 5, 7 days delay of nerve growth factor treatment was ineffective in attenuating cognitive deficits.

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Various publications are cited herein, the contents of which are hereby incorporated, by reference, in their entireties.

What is claimed:

1. A method for reducing the cognitive, sensory or motor deficits associated with traumatic injury to the central nervous system comprising administration of an effective amount of nerve growth factor to a subject whose nervous system has been damaged by trauma.

2. The method of claim 1 wherein the region of the nervous system damaged by trauma is the brain.

3. The method of claim 1 wherein the region of the nervous system damaged by trauma is the spinal cord.

4. The method of claim 1 wherein the nerve growth factor is administered intraventricularly.

5. The method of claim 1 wherein the nerve growth factor is administered into the basal forebrain, hippocampus or subarachnoid space.

6. The method of claim 1 wherein the nerve growth factor is administered in the subarchnoidord space in close proximity to the site of injury.

7. The method of claim 1 further comprising the administration of a second neurotrophic reagent.

8. The method of claim 7 wherein the second neurotrophic reagent is selected from the group consisting of brain derived neurotrophic factor, neurotrophin-3, ciliary neurotrophic factor, or glial-derived neurotrophic factor.

9. The method of claim 1 wherein the dose of nerve growth factor is adjusted depending on changes in ventricular size or cerebrospinal fluid volume.

10. The method of claim 1 wherein the daily dose of nerve growth factor is between 150 μg and 4 mg.

11. The method of claim 1 wherein the daily dose of nerve growth factor is between 250 μg and 3 mg.

12. The method of claim 1 wherein the daily dose of nerve growth factor is between 500 μg and 1.5 mg.

13. The method of claim 1 wherein the nerve growth factor is administered between 4 and 8 days post-injury.

14. The method of claim 1 wherein the nerve growth factor is administered immediately after injury and up to at least 28 days post-injury.

15. The method of claim 1 wherein the nerve growth factor is administered for between at least 10 and 112 consecutive days.

16. The method of claim 1 wherein the nerve growth factor is administered for between at least 20 and 54 consecutive days.

17. The method of claim 1 wherein the nerve growth factor is administered for at least 28 consecutive days.

18. The method of claim 1 wherein the nerve growth factor is delivered by infusion into the central nervous system.

19. The method of claim 1 wherein the nerve growth factor is delivered by injection into the central nervous system.
